(19) United States
(12) Patent Application Publication
(10) Pub. No.: US 2012/0265155 A1
(43) Pub. Date: Oct. 18, 2012

(54) APPARATUS AND METHODS FOR PROVIDING A MEDICINAL SUBSTANCE APPLICATOR

(76) Inventor: Tomas Baez, Davie, FL (US)

(21) Appl. No.: 13/088,350

(22) Filed: Apr. 16, 2011

(51) Int. Cl. A61M 5/00; B23P 11/00

(52) U.S. Cl. 604/263; 29/428

(57) ABSTRACT

Embodiments include a method of providing a medicinal substance applicator that includes attaching an absorbent material at an end of a needle cap.

### FIELD

This written description is in the field of apparatuses and methods of providing a medicinal substance applicator.

### BRIEF DESCRIPTION OF THE DRAWINGS

Aspects of embodiments will become apparent upon reading the following detailed description and upon reference to the accompanying drawings in which like references may indicate similar elements:

FIG. 1 depicts a needle with a needle cap and an absorbent material applied to the needle cap;

FIG. 2 depicts a needle cap having a first circumference and a second circumference and an absorbent material applied to the second circumference;

FIG. 3 depicts a needle cap with an absorbent material applied to an end of the needle cap;

FIG. 4 depicts a needle cap with a tapered end to receive an absorbent material that may be covered with a cover.

### DETAILED DESCRIPTION

The following is a detailed description of embodiments depicted in the accompanying drawings. The amount of detail offered is not intended to limit the anticipated variations of embodiments; but, on the contrary, the intention is to cover modifications, equivalents, and alternatives falling within the scope of the appended claims. The detailed descriptions below are designed to make such embodiments obvious to a person of ordinary skill in the art.

Embodiments include a method of providing a medicinal substance applicator that includes attaching an absorbent material at an end of a needle cap. The end of the needle cap with the absorbent material may be applied to a body. In one embodiment, the needle cap is a dental needle cap, the surface of the body is a soft tissue inside a mouth, and the medicinal substance is a topical anesthetic. Thus, the needle cap with the absorbent material is of a size and length to enable the needle cap with absorbent material to be inserted into a mouth to apply the topical anesthetic on the soft tissue where the needle will be inserted. In another embodiment, the medicinal substance is a disinfectant or antiseptic that is applied to the body. For example, before the needle covered by the needle cap is used to draw blood, alcohol applied to absorbent material at the end of the needle cap is applied to the skin where the needle is to be injected. This eliminates a need to use a separate ball of cotton, for example.

Another embodiment is an apparatus for applying a medicinal substance to a body. The apparatus includes a needle cap. Positioned on the needle cap is an absorbent material to absorb a medicinal substance so that the absorbent material on the needle cap may be applied to the body. In some embodiments, the absorbent material is applied directly to the needle cap. In other embodiments, the absorbent material is affixed to an auxiliary part that is affixable to the needle cap.

Another embodiment is a method of providing a medicinal substance applicator. The method includes packaging a plurality of needles in a package, each needle being covered by a needle cap. Included in the package is, for each of one or more needle caps, an absorbent material that is one of being affixed and affixable to a needle cap. The package may include a receptacle having a medicinal substance into which an absorbent end of a needle cap may be inserted or dipped to absorb at least a portion of the medicinal substance. In some embodiments the medicinal substance is a topical anesthetic. In other embodiments, the medicinal substance is an antiseptic.

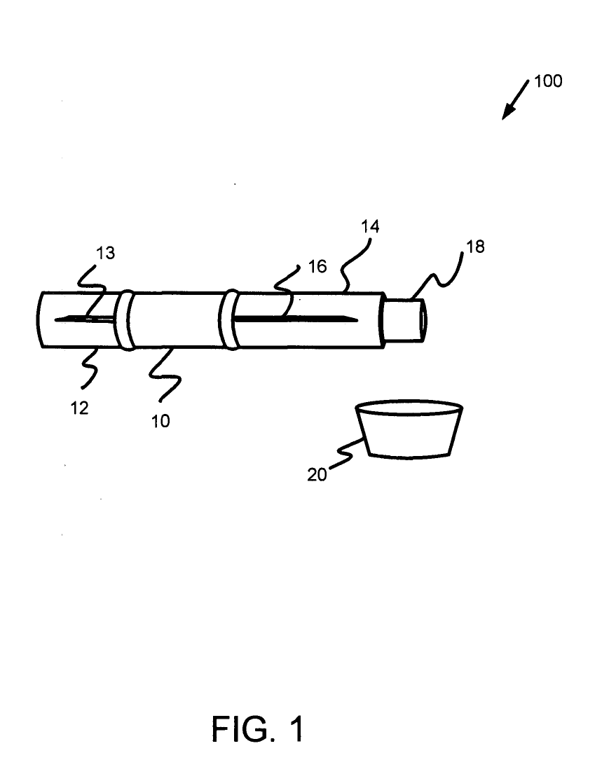

FIG. 1 shows a needle assembly 10 with a first needle cap 12 that covers a first end 13 of a needle and a second needle cap 14 that covers a second end 16 of the needle. When the needle cap 12 is removed, the first needle end 13 may be applied to puncture a cap in a carpule that provides a medication, such as an anesthetic, to be injected into a body. When the needle cap 14 is removed, the second needle end 16 may be inserted into a body to apply the medication in the carpule. As is known in the art of dentistry, for example, a carpule is a type of ampule or cartridge that may contain liquid anesthetic to be inserted into the soft tissue using a syringe.

Typically, a plurality of needle assemblies 10, are packaged in a box, and sold by the box to, for example, a dental office. In the dental setting, for example, a needle assembly is used to inject an anesthetic into the mucosa of the mouth of a patient to numb the surrounding soft tissue, and nerves of teeth prior to dental work being performed on the teeth. For example, a dentist may remove the first needle cap 12 and insert the first needle end 13 into a carpule having the anesthetic. The dentist will also remove the second needle cap 14 and insert the second needle end 16 into a patient's soft tissue to inject the anesthetic from the carpule.

In some cases, a dentist may apply a topical anesthetic to the soft tissue (mucosa) before injecting an anesthetic into the soft tissue with a needle. The topical anesthetic is usually applied using a prior art applicator that consists of a stick or shaft with an absorbent material at an end that has been dipped into a topical anesthetic. An advantage of the present invention is that a separate applicator is not needed because the needle cap with absorbent material provides an applicator for applying the topical anesthetic. Thus, a separate box of applicators need not be ordered and stored.

According to one aspect, an absorbent material 18 is affixed at an end of the needle cap 14. The absorbent material may include sponge, fibers or cotton, for example. The end of the needle cap that has the absorbent material may be dipped into a disposable dish 20 containing the medicinal substance to be applied to the body. The disposable dish 20 is designed to provide a single dose of the medicinal substance to a single patient to prevent cross contamination. Thus, the dish 20 may be a uni-dose dish that will be sealed to preserve the substance to be applied. In some embodiments, the dish 20 is one of one or more receptacles packaged in the box with the plurality of needles. The needles packaged in the box may have the absorbent material already attached or affixed upon the needle cap of each needle. Alternatively, the absorbent material may be in the form of units packaged with the needles but separated from the needle caps, leaving it to the health care provider to place the unit of absorbent material upon the needle cap. In some embodiments, the medicinal substance may be applied to the absorbent material before the needle assembly is enclosed in a package.

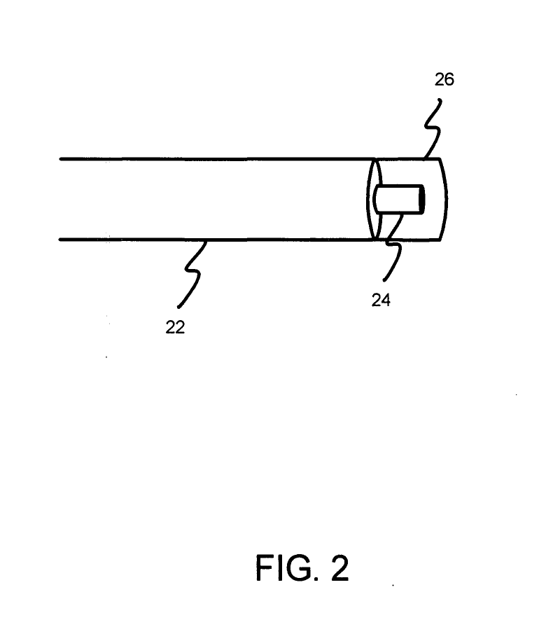

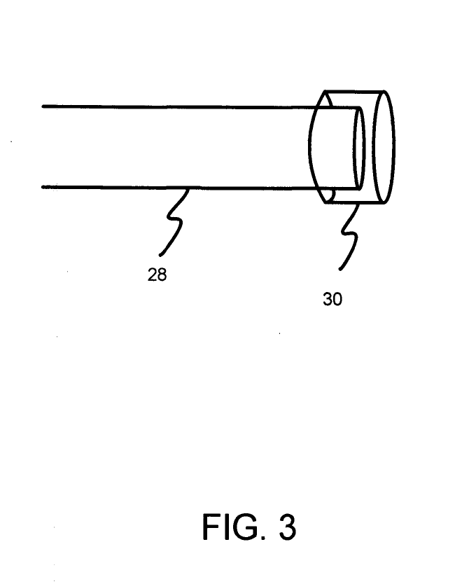

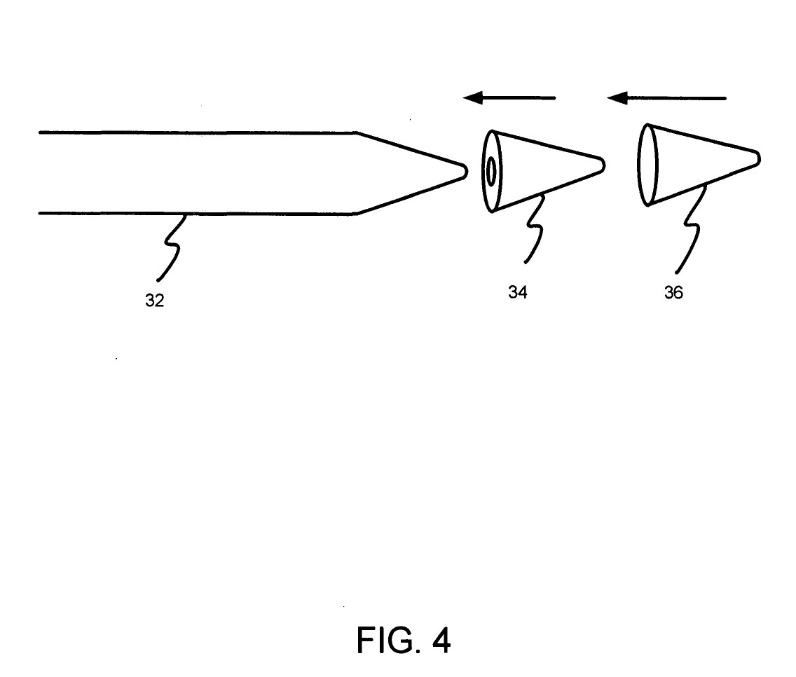

Other exemplary embodiments are shown in FIGS. 2-4. For example, FIG. 2 depicts a needle cap 22 having a first circumference and a second circumference 24 and an absorbent material 26 applied to the second circumference. In some embodiments, the absorbent material is pre formed and slides over the part 24. The absorbent material may be removably applied to the part 24 or may be affixed with adhesive to the part 24. FIG. 3 depicts a needle cap 28 that receives an end cap 30 comprising an absorbent material to absorb a medicinal substance such as a topical anesthetic. The end cap 30 may be made of sponge or cotton, for example, and may be pre-formed to be removably affixed or irremovably affixed to the needle cap 28. FIG. 4 shows another embodiment that includes a needle cap 32 that is tapered along its length to receive an absorbent material 34. In some embodiments, the absorbent material 34 may be covered by a removable plastic cover 36. In other embodiments, the length of the needle cap to which the absorbent material is applied may vary and the length of the portion of the needle cap that is covered by absorbent material may vary.

Thus, in some embodiments, the needle cap with the absorbent material is of a size and length to enable the needle cap with absorbent material to be inserted into the mouth of a dental patient to apply a topical anesthetic to the soft tissue. The absorbent material may cover the end of the needle cap and may also partially cover the sides of the needle cap. In some embodiments, the absorbent material may be affixed to an auxiliary part that is affixable to the needle cap. In some embodiments, the absorbent material is spherical, pyramidal, square or cylindrical in shape. Further, in some embodiments, the absorbent material covers substantially the entire length of the needle cap.

Various changes, substitutions and alterations can be made to the embodiments described herein without departing from the scope of the appended claims. An embodiment may achieve multiple objectives, but not every embodiment falling within the scope of the attached claims will achieve every objective. Moreover, the scope of the present application is not intended to be limited to the particular embodiments of the process, machine, manufacture, composition of matter, means, methods and steps described in the specification. One of ordinary skill in the art will readily appreciate from this disclosure that processes, machines, manufacture, compositions of matter, means, methods, or steps, presently existing or later to be developed are equivalent to, and fall within the scope of what is claimed. Accordingly, the appended claims are intended to include within their scope such processes, machines, manufacture, compositions of matter, means, methods, or steps.

What is claimed is:

1. A method of providing a medicinal substance applicator, comprising:
attaching an absorbent material at an end of a needle cap, so that a medicinal substance can be applied to the absorbent material and so that an end of the needle cap with the absorbent material may be applied to a body.

2. The method of claim 1, wherein the needle cap with the absorbent material is of a size and length to enable the needle cap with absorbent material to be inserted into a mouth to apply the medicinal substance to a soft tissue and wherein the medicinal substance is a topical anesthetic.

3. The method of claim 1, wherein the absorbent material covers the end of the needle cap and partially covers a side of the needle cap.

4. The method of claim 1, further comprising packaging the needle cap with the absorbent material.

5. An apparatus for applying a medicinal substance to a body, comprising:
a needle cap; and
positioned on the needle cap, an absorbent material to absorb the medicinal substance so that the absorbent material on the needle cap may be applied to the body.

6. The apparatus of claim 5, wherein the absorbent material is affixed to an auxiliary part that is affixable to the needle cap.

7. The apparatus of claim 5, wherein the absorbent material is removably affixable to the needle cap.

8. The apparatus of claim 5, wherein the absorbent material partially covers a side of the needle cap.

9. The apparatus of claim 5, wherein the absorbent material covers the end of the needle cap.

10. The apparatus of claim 5, wherein the medicinal substance is an antiseptic.

11. The apparatus of claim 5, wherein the absorbent material is a sponge.

12. The apparatus of claim 5, wherein the absorbent material includes cotton.

13. The apparatus of claim 5, wherein the needle cap exhibits a circumference that is tapered along a length of the needle cap.

14. The apparatus of claim 5, wherein the needle cap exhibits a first circumference and a second circumference smaller than the first circumference and wherein the portion of the needle cap having the second circumference receives the absorbent material.

15. A method of providing a medicinal substance applicator, comprising:
packaging a plurality of needles in a package, each needle being covered by a needle cap; and
including in the package for each of one or more needle caps, an absorbent material that is one of being affixed and affixable to a needle cap.

16. The method of claim 15, further comprising including in the package, at least one receptacle having a medicinal substance.

17. The method of claim 16, wherein the receptacle is a dish into which an absorbent material at an end of a needle cap may be inserted to absorb at least a portion of the medicinal substance.

18. The method of claim 15, wherein the medicinal substance is applied to the absorbent material before the needles are enclosed in the package.

19. The method of claim 15, wherein the medicinal substance is alcohol to be applied to the body.

20. The method of claim 15, wherein the medicinal substance is a topical anesthetic.
